(19) United States
(12) Patent Application Publication
(10) Pub. No.: US 2003/0146111 A1
(43) Pub. Date: Aug. 7, 2003

(54) Enzymatic-electrochemical measuring device

(76) Inventors: Peter Abel, Zuessow (DE); Thomas Von Woedtke, Horst (DE); Alexander Schwock, Griebenow (DE)

Correspondence Address: JORDAN AND HAMBURG LLP, 122 EAST 42ND STREET, SUITE 4000, NEW YORK, NY, 10168, US

(21) Appl. No.: 10/220,169

(22) PCT Filed: Feb. 28, 2001
(86) PCT No.: PCT/DE01/00824

(30) Foreign Application Priority Data
Feb. 28, 2000 (DE) 100 09 467.8

(51) Int. Cl. C12Q 1/54

(52) U.S. Cl. 205/777.5

(57) ABSTRACT

The invention relates to an enzymatic-electrochemical measuring device, based on a Clark electrode, comprising an Ag/AgCl reference electrode as the anode and an electrochemically active working electrode, said electrodes being configured from a layer of electrolyte, a hydrophobic membrane and an enzyme as a sensor. The invention is characterised in that at least two of the sensors are arranged as dual sensors, the electrodes of both sensors are wetted in a uniform manner and covered by a layer of electrolyte and a membrane with hydrophobic properties, the hydrophobic membrane is a predominantly gas-permeable polymer, on which, in the case of the working electrode, an enzyme is immobilised, whereas the hydrophobic membrane of the other sensor is devoid of enzymes. The measuring device which can be commercially produced and is easy to use is used for determining, controlling and monitoring the presence and concentration of different analytes in medical diagnostics and in the food and biotechnological industries. The measuring device is particularly advantageous for determining the level of urinary glucose both in a stationary and an ambulant environment and can also he used by the elderly in their homes.

The invention is for an enzymatic-electrochemical measuring device with Clark electrodes with a wide scope of applications beyond medical diagnosis.

To control metabolic situations, especially those of diabetics, requires that the circulating glucose concentration be checked regularly. This is true not only for insulin-injecting Type 1 diabetics, but for Type 2 diabetics as well, whereby the latter account for about 85% of the cases of diabetes. Worldwide, the number of diabetics is predicted to reach 221 million by 2010 (Hauner, H.; Dtsch Med Wochenschr 1998; 123; 777-82; Pharmaco Economics 1995; 8 (Suppl. I) 28-71; Ammon, H. P. T.; Deutsche Apoteker Zeitung; 1999; 139 Jahrgang Nr. 30).

Measured on the high number of diabetic patients, the only routine method of measuring blood sugar using penetration of the skin cannot fulfill the demand or the desire for regular metabolic checks. This is based on both the difficulty of overcoming the psychic hurdle of damaging the skin prior to every measurement as well as material expense required for completing any measurement except for the use of disposable materials.

For the majority of those effected a blood sugar measurement is not even indicated, but rather checking the presence of glucose in the urine is sufficient. Glucose in the urine is detectable in concentrations if the renal threshold is exceeded by irritation of the metabolism so that in addition to the physiological utilization of glucose it is also excreted as urinary sugar by the kidneys.

A recognized method of determining the urinary sugar is based on the property of glucose to rotate polarized light to the right. This property is used in the polarimeter, whereby the angle of deviation of the light from the polarization plane represents the measure of the glucose concentration (Pöge, A. W. Z. med. Labortechnik 17 (1976) 59-78).

Urinary sugar test strips are used especially for preventive screening measures; this method generally generates a glucose-dependent color reaction based on enzymatic-chemical reactions. These test strips have the disadvantage of being very inaccurate and therefore unreliable results and can only be analyzed by persons good with colors. Especially for diabetics, especially the older Type 2, a large number are colorblind or at least uncertain of colors.

While the polimeter has established itself as a laboratory device in clinical chemistry, urinary sugar test strips are not suitable as a routine method for a reliable determination of glucose and its concentration in daily metabolic checks.

Research and development are therefore concentrating on enzymatic-electrochemical measuring devices and their application for determining glucose.

The electrochemical basic sensor of the enzymatic-electrochemical biosensor and corresponds to the classical Clark oxygen sensor is known and has often been described in literature.

A definition of the enzymatic electrochemical biosensor for determining analytes in liquids is therefore revealed in DD 227 029 A3:

According to this technical solution for measuring glucose the enzyme-electrode consists of a sandwich membrane structure which has a one hydrophobic and one hydrophilic membrane with an enzyme located between them.

This arrangement takes advantage of the modified classical Clark electrode exclusively for the determination of concentration of analytes to be detected resulting form an enzymatic oxidation using the hydrogen peroxide generated when oxygen is used. The hydrogen peroxide is electrochemically oxidized on the anode at a potential of 600 mV to 700 mV. This arrangement has the disadvantage that other electrochemically active substances in the sample, which can also pass through the membrane system, also enter into the reaction layer of the biosensor and may be oxidized on the working electrode just like the and hydrogen peroxide and may then, as interfering substances, lead to a falsification of the measurement signal.

Furthermore, such substances forming on the working electrode may influence the electrode in its electrochemical properties and result in an electrode contamination and aging.

The interfering effect of the non-specific analytes be reduced by selecting a suitable electrode potential between the working and the reference electrode in connection with a mediator which acts in place of the oxygen as the electron acceptor, whereby the reduction of the electrode potential to about 300 mV is possible. The general access of foreign molecules to the electrode system does, however, remain unavoidable with this system.

The disadvantages mentioned result in the measurements being influenced by the interfering substances or electrode contamination or aging to such a degree that the desired statements on the presence of the actual analytes to be detected are not certain, stable or reproducible.

The task of the invention is therefore to provide a reuseable, improved device based on enzymatic-electochemistry with which it is possible to definitely determine and display the presence and concentration of analytes in aqueous solutions in an easy and reproducible manner. In particular it is the objective of the device to make it possible for elderly people under normal daily conditions, using routine examinations to measure the presence and concentration of glucose in urine and display and/or signal this in a suitable manner.

The invention attains the objective by means of an enzymatic-electrochemical device with the characteristics of claim 1. Advantageous aspects of the invented measuring device are to be found in the characteristics of the sub-claims 2 to 8. The use of the invented measuring device are found in the characteristics of the claims 9 and 10.

The invented measuring device has the advantage that it can be used in a variety of ways for determination, control and monitoring of the presence and concentration of various analytes in medical diagnostics and in food technology and biotechnological industries. It is based on an enzymatic-electrochemical biosensor formed as a double sensor applying the renowned principle of a Clark oxygen electrode in connection with a blood vessel that is suitable for the recording of the dual biosensor and a sufficient sample volume.

For glucose determination the sample vessel is formed as an overflow vessel that retains a sufficient amount of urine in the sensitive area of the biosensor. That the vessel accepting the dual enzymatic-electrochemical biosensor is constructed in a manner that it can take up a defined and definite portion of the urine for the specific measurement during the excretion process. It is designed so that the portion of the vessel in which the dual enzymatic-electrochemical biosensor for glucose is inserted is filled to overflowing while the excessive volumes can flow off.

In the following the function of the invented measuring device and its advantages in relation to the state of the art will be explained using the example of determining glucose in urine, without limiting the device in its scope.

From the state of the art technology (DD 227 029 A3) it is known that physically dissolved oxygen passes through a hydrophobic membrane that is only gas-transparent and placed before the classical Clark electrode and is electrochemically reduced at a working electrode. Here two electrons are released for each oxygen molecule, generating an electric current which represents the current concentration of the physically dissolved oxygen, the oxygen partial pressure.

In the case of the equally renowned enzymatic-electrochemical biosensor for glucose, an enzymatic layer, which should contain the enzyme glucose oxidase is covered by a hydrophilic membrane which is permeable for ions and molecules that are released, is placed in front of the hydrophobic membrane. Released from the solution in which the sensor is located, an analyte, whose oxidation is catalyzed by the enzyme present, the oxygen partial pressure at the working electrode is reduced as a result of the oxygen consumption in this reaction. This means that that in this case the measured oxygen partial pressure is reduced by the sum that is used by the enzymatic reaction. In this way the difference between the original oxygen partial pressure in the solution and the specific contribution of the reduced oxygen partial pressure at the working electrode is a measure of the concentration of the analyte to be detected. The evaluation of this partial pressure difference does, however, require that the original oxygen partial pressure in the medium to be analyzed be known and constant. This prerequisite is, however, not met in biological solutions. This is where the invention is applied.

To be able to use the measurement of the oxygen consumption as an indicator for the currently available glucose in the sample to be examined, the known electrochemical basic sensor has been further developed and arranged in the manner of the invention in that an additional sensor has been added to the classical Clark electrode system; the hydrophobic membrane covering of the working electrode is not coated with the enzyme for the oxidation of the analyte to be detected. The oxygen at this working electrode is reduced according to its current concentration in the sample to be analyzed, because there is no oxygen consumption caused by an enzymatic reaction. The signals of the working electrodes of the dual sensor formed in this manner are, in accordance with the invention, led into a difference amplifier in which a signal difference is formed in case the analyte glucose is present. The further processing of this specific measurement signal occurs optionally in a method preferred by the user.

The invented solution guarantees the advantage of a complete, but nevertheless selective, exclusion of the substances influencing the measurement of the working electrode, on the one hand, is combined with an equally complete and selective access of the analyte to be detected or of a product of a specific detection reaction at the working electrode, on the other hand. Interferences and an electrode contamination are excluded in this manner.

The dual enzymatic-electrochemical biosensor and a overflow vessel large enough to hold a sufficient volume of urine for the corresponding measurement can be designed according to the invention for a device that is either portable or for the bathroom sink.

In this way the industrially produced and easy to use solution for urinary sugar determination is suitable for hospital or ambulatory use and therefore can be used by elderly persons at home for daily use. The number of possible measurements is limited only by the lifespan of the biosensor.

If the glucose oxidase enzyme is replaced by another oxygen-consuming reaction partner, other analytes can also be determined in media of unknown composition. There is, for instance, the possibility of using lactate oxidase as enzyme for measuring and displaying the concentration of lactate in sugar beet juice or other liquids.

1. Enzymatic-electrochemical measuring device based on a Clark electrode consisting of an Ag/AgCl reference electrode as anode and an electrochemically active working electrode, said electrodes being configured from a layer of electrolyte, a hydrophobic membrane and an enzyme as a sensor, characterized by at least two of the sensors being arranged as dual sensors, the electrodes of both sensors are covered by an electrolyte layer and a membrane with hydrophobic properties, the hydrophobic membrane is a predominantly gas permeable polymer, on which, in the case polymer, on which, in the case of working electrode, an enzyme is immobilized, whereas the hydrophobic membrane of the other sensor is devoid of enzymes.

2. Enzymatic-electrochemical measuring device in accordance with claim 1, characterized by the electrochemical working electrode being a gold, platinum, carbon electrode or an electrode made of an oxygen-active catalyst, preferably a ruthenium-selenium or iron basis.

3. Enzymatic-electrochemical measuring device in accordance with claim 1 or 2, characterized by the enzyme being an oxygen-consuming enzyme, an oxidase, preferably glucose-oxidase, lactate-oxidase, alcohol-oxidase, sulfite-oxidase, urease or a peroxidase.

4. Enzymatic-electrochemical measuring device in accordance with one of the claims 1 to 3, characterized by the enzyme-covered electrode being formed by coupling various enzymes as bi- or multi-enzyme electrodes, preferably by coupling enzymes from the oxidase group and enzymes from the peroxidase group.

5. Enzymatic-electrochemical measuring device in accordance with one of the claims 1 to 4, characterized by the enzyme-covered membrane being covered by a hydrophilic membrane.

6. Enzymatic-electrochemical measuring device in accordance with one of the claims 1 to 5, characterized by the sensor that does not have an enzyme measuring the original, undisturbed oxygen partial pressure of the sample solution and the enzyme-coated sensor oxygen partial pressure reduced by the enzymatic reaction.

7. Enzymatic-electrochemical measuring device in accordance with one of the claims 1 to 6, characterized by the signals of the signals of the two sensors being entered in a difference amplifier and the difference amplifier making the difference of the two electrode signals as a qualitative and quantitative indicator for the presence or absence of the analyte to be detected or its concentration and displaying these vales either optically or acoustically, in analog or digital form.

8. Enzymatic-electrochemical measuring device in accordance with one of the claims 1 to 7, characterized by the sensors being arranged in an overflow vessel and the overflow vessel being designed so that a sufficient filling volume is guaranteed for each measurement.

9. Use of the enzymatic-electrochemical measuring device in accordance with one of the claims 1 to 8 for determining, controlling and monitoring the presence and concentration of glucose in urine and the determining, controlling and monitoring of the presence and concentration of glucose, lactate, uric acid, sulfite, maltose, saccharine, glutamate, fructose, pyruvate, phenolene, glutamine, galactose and lysine.

10. Use in accordance with claim 9, characterized by the enzymatic-electrochemical measuring device for determining, controlling and monitoring the presence and concentration of glucose in urine is designed for use in bathroom sinks and/or urinals in hospitals or that it is portable.
Summary
The invention relates to an enzymatic-electrochemical measuring device, based on a Clark electrode, comprising an Ag/AgCl reference electrode as the anode and an electrochemically active working electrode, said electrodes being configured from a layer of electrolyte, a hydrophobic membrane and an enzyme as a sensor, whereby at least two of the sensors are arranged as dual sensors, the electrodes of both sensors are covered by an electrolyte layer and a membrane with hydrophobic properties, the hydrophobic membrane is a predominantly gas permeable polymer, on which, in the case polymer, on which, in the case of working electrode, an enzyme is immobilized, whereas the hydrophobic membrane of the other sensor is devoid of enzymes.
The measuring device which can be industrially produced and is easy to use is used for determining, controlling and monitoring the presence and concentration of different analytes in medical diagnostics and in the food and biotechnological industries.
The measuring device is particularly advantageous for determining the level of urinary glucose both in hospital and out-patient conditions and can also be used by the elderly in their homes.
